(19) United States
(12) Patent Application Publication
(10) Pub. No.: US 2026/0223870 A1
(43) Pub. Date: Aug. 6, 2026

(54) COMPOUNDS

(71) Applicant: GIVAUDAN SA, VERNIER (CH)

(72) Inventors: Lise LAURENCON, Garlin (FR); Laure ZIEGLER, Avignon (FR); Simona BIRTIC, Avignon (FR)

(21) Appl. No.: 19/152,539

(22) PCT Filed: Feb. 1, 2024
(86) PCT No.: PCT/EP2024/052499

§ 371 Date: Jul. 31, 2025

(30) Foreign Application Priority Data
Feb. 3, 2023 (GB) 2301565.4

(51) Int. Cl. A23B 2/75; A23B 4/20; A23B 70/10; A61K 8/34; A61K 8/37; A61K 36/53; A61K 36/74; A61K 47/10; A61K 47/14

(52) U.S. Cl. CPC A23B 2/75; A23B 4/20; A23B 70/10; A61K 8/347; A61K 8/375; A61K 36/53; A61K 36/742; A61K 47/10; A61K 47/14; A61K 2800/522

(57) ABSTRACT

The present invention relates to a composition with synergistic antioxidant effect whose components are obtained from plant extracts, wherein the composition is useful in the preparation of foodstuffs.

### TECHNICAL FIELD OF THE INVENTION

The present invention relates to the use of plant extracts to obtain antioxidants which are useful in the preparation of foodstuffs, cosmetic products and nutraceuticals.

### BACKGROUND OF THE INVENTION

Antioxidants are compounds that inhibit oxidation, a chemical reaction that can produce free radicals and chain reactions that may damage the cells of organisms. Antioxidants are widely used in the agro-industrial industry, namely in meat and poultry, beverages, salad dressings, seasonings, snacks, etc., to preserve food by retarding the deterioration, rancidity or discoloration of food. In addition, antioxidants have also been used in various cosmetics and toiletry products to prevent oxidation, as well as in pharmaceutical compositions. Finally, antioxidants are also commonly used as nutraceuticals. The primary use of antioxidants is therefore the prevention of deterioration of product characteristics by preventing oxidation damage as well as preventing cell damage due to oxygen radicals.

The use of synthetic antioxidants in commercial food preparation has mostly been replaced by natural sources of antioxidants, such as those found on spices, such as rosemary and sage. Antioxidants obtained from natural sources have been shown to be stable at high temperatures, are readily soluble in oil and are considered to be safer than synthetic antioxidants. Most of the natural antioxidants are derived from plant materials, such as fruit, vegetables, herbs and spices.

Antioxidants are mostly obtained from liquid extracts in solvent of the natural sources wherein the antioxidants are present. Small changes in the process of obtaining the plants and or treatment leading up to the extraction process can lead to major output differences in yield and as well as in the antioxidant activity of the extract. Furthermore, scale-up of such extractions is not always easy and leads to an increase in the production of by-products. Therefore, in order to facilitate the extraction process of antioxidants and help reducing the amount of by-product from the extraction process, alternative antioxidants with higher antioxidant capacity are necessary.

### SUMMARY OF THE INVENTION

The inventors have discovered that certain combinations of antioxidant extracts lead to a synergistic effect in the antioxidant activity of the extract composition.

Therefore a first aspect of the present invention relates to an antioxidant composition comprising chlorogenic acid and one or more additional component or combination of components selected from a group consisting of:
(i) rosmarinic acid; and
(ii) phenolic diterpenes,
   wherein the ratio of chlorogenic acid to the one or more additional component or combination of components is of between about 100:1 to about 1:100.

Another aspect of the present invention relates to the use of the composition according to the invention in the manufacture of food or beverage product for humans or animals, a nutritional supplement, a nutraceutical formulation, a fragrance or flavouring, a pharmaceutical or veterinary formulation, an oenological or cosmetic formulation.

Another aspect of the present invention relates to the use of the composition according to the invention as an antioxidant.

One more aspect of the present invention relates to a process for preventing and/or inhibiting oxidation of a food or beverage product for humans or animals, a nutritional supplement, a nutraceutical formulation, a fragrance or flavouring, a pharmaceutical or veterinary formulation, an oenological or cosmetic formulation, the process comprising the steps of contacting the foodstuff with the composition according to the invention.

One more aspect of the present invention relates to a food or beverage product for humans or animals, a nutritional supplement, a nutraceutical formulation, a fragrance or flavouring, a pharmaceutical or veterinary formulation, an oenological or cosmetic formulation comprising the antioxidant composition according to the invention or a nutraceutical or dietary supplement comprising the composition according to the invention and a food or beverage product for humans or animals, a nutritional supplement, a nutraceutical formulation, a fragrance or flavouring, a pharmaceutical or veterinary formulation, an oenological or cosmetic formulation acceptable adjuvant.

### BRIEF DESCRIPTION OF THE FIGURES

FIG. 1. ORAC-CAT Values for a blend containing a rosemary and green coffee beans.

FIG. 2. CAT Values for a blend containing sage and green coffee beans.

FIG. 3. TBArs values during TSL=10 days in pork meat.

### DETAILED DESCRIPTION OF THE INVENTION

As previously mentioned the present invention discloses compositions of extracts with antioxidant effect that present a synergistic antioxidant activity in comparison with the antioxidant activity of the individual extracts. These compositions are obtained by mixing in certain proportions antioxidant extracts derived from different plant sources.

### Compositions of the Invention

As such, a first aspect of the present invention relates to an antioxidant composition, from here onwards the composition of the invention, comprising chlorogenic acid and one or more additional component or combination of components selected from a group consisting of:
(i) rosmarinic acid; and
(ii) phenolic diterpenes,
   wherein the ratio of chlorogenic acid to the one or more additional component or combination of components is of between about 100:1 to about 1:100.

The term “antioxidant” as used herein refers to compounds that are capable of inhibiting reactions promoted by oxygen or induced by the presence of metal, such as iron (reaction of Fenton), thus avoiding oxidation and rancidity of the compositions. An antioxidant therefore reduces the amount of oxidation over a given period when compared to the oxidation that would occur in its absence. Methods to determine the antioxidant capacity of the compound of a composition are well known in the art and are exemplified in the Examples section of the description under the sections “In vitro ORAC test” and “In vitro CAT test”.

The term “chlorogenic acid” as used herein refers to the ester of caffeic acid and (−)-quinic acid. Chlorogenic acid is naturally present in coffee, mainly as mono- and di-esters of quinic acid and phenolic groups (e.g. caffeic, ferulic, coumaric, methoxycinnamic) attached to different positions. Chlorogenic acid is defined in the entry of the database CAS Registry with the number 202650-88-2 as well as structurally by formula (iii)

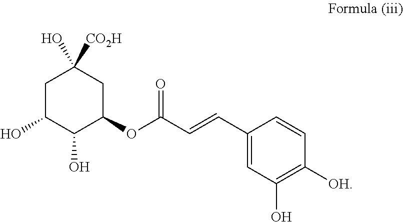 [SMILES: O=C(/C=C/c1ccc(O)c(O)c1)O[C@@H]1C[C@](O)(C(=O)O)C[C@@H](O)[C@H]1O]

The ratio of the chlorogenic acid can be in relation to one additional component of the composition of the invention, such as rosmarinic acid or phenolic diterpenes, or it can be in relation to both additional components of the composition of the invention, ie., rosmarinic acid and phenolic diterpenes. In a preferred embodiment of the composition of the invention the ratio of chlorogenic acid to the one or more additional components or combination of components is of about 95:5, 97:7, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 7:97, about 5:95. In a preferred embodiment of the composition of the invention the ratio of chlorogenic acid to the one or more additional components or combination of components is of between 50:1 to 1:50, preferably between 25:1 to 1:25. In a preferred embodiment of the composition of the invention the ratio of chlorogenic acid to the one or more additional components or combination of components is of between about 50:1 to about 1:50, such as between about 25:1 to about 1:25, such as between about 10:1 to about 1:10, such as between about 5:1 to about 1:5.

In a preferred embodiment of the composition of the invention the concentration of chlorogenic acid is of between about 0.1% w/v to 55% w/w, about 0.5% to about 25%, about 1% to about 10%, about 2.0% to about 3.0%, about 2.1% w/w to 2.9% w/w, about 2.15% w/w to 2.85% w/w, about 2.2% w/w to 2.8% w/w, about 2.25% w/w to 2.75% w/w, about 2.3% w/w to 2.7% w/w, about 2.3% w/w to 2.65% w/w, about 2.3% w/w to 2.6% w/w, about 2.3% w/w to 2.55% w/w, about 2.3% w/w to 2.5% w/w, about 2.3% w/w to 2.45% w/w, about 2.3% w/w to 2.4% w/w, about 2.3% w/w to 2.4% w/w. In a more preferred embodiment of the composition of the invention the concentration of chlorogenic acid is of between about 4% to about 5%, of between about 2% w/w to about 3% w/w.

The composition of the invention further comprises one or more additional components selected from rosmarinic acid and phenolic diterpenes.

The term “diterpenes” as used herein refers to a class of chemical compounds composed of four isoprene units, often with the molecular formula C₂₀H₃₂. They are biosynthesized by plants, animals and fungi via the HMG-CoA reductase pathway, with geranylgeranyl pyrophosphate being a primary intermediate. “Phenolic diterpenes” are a subgroup of diterpenes which include, among others, carnosic acid, carnosol, rosmanol, epirosmanol and methoxyepirosmanol. These components are, at least to some extent, the result of the degradation of carnosic acid but they still possess antioxidant activity. “Phenolic diterpenes” are meant to include carnosic acid, carnosol, and their derivatives. Therefore, when one refers to “phenolic diterpenes” in the present invention, all of carnosic acid, carnosol and their derivatives are included.

In a preferred embodiment of the composition of the invention, the ratio of chlorogenic acid to phenolic diterpenes is of between about 25:1 to about 1:25, between about 10:1 to about 1:10, between about 5:1 to about 1:5, preferably between about 0.9:1 to about 1:0.9.

In a preferred embodiment of the composition of the invention the concentration of the phenolic diterpenes is of at least about 0.5% w/w, at least about 1.7% w/w, at least about 1.8% w/w, at least about 1.9% w/w, at least about 2.0% w/w, at least about 2.1% w/w, at least about 2.2% w/w, at least about 2.3% w/w, at least about 2.4% w/w, preferably at least about 2.5% w/w, at least about 3.0% w/w, at least 5.0% w/w, at least about 10% w/w, at least about 25% w/w, at least 50% w/w, at least 90% w/w.

The phenolic diterpenes of the composition of the present invention can be obtained as an extract of Salvia aerial parts. In a preferred embodiment of the composition of the invention, the phenolic diterpenes are provided as an extract of Salvia aerial parts, preferably leaves. “Salvia aerial parts” refers to stems, leaves, petioles, flowers, fruit and seeds, preferably leaves, from the genus of plants in the sage family Lamiaceae, composed of shrubs, herbaceous perennials and annuals. In a preferred embodiment of the composition of the invention the Salvia aerial parts, preferably leaves, are from the plant Salvia officinalis or from the plant Salvia apiana.

The person skilled in the art will be aware of known methods for providing an extract of Salvia aerial parts, since these are well known in the art. For example the methods described in the document WO96/34534, which is incorporated here by reference, may be used in the present invention. In a preferred embodiment of the composition of the invention the extract of Salvia aerial parts, preferably leaves, is obtained by:
(i) extracting the Salvia aerial parts, preferably leaves, with acetone and activated carbon; and optionally
(ii) further purifying the extract.

In step (i) of the method to obtain an extract of Salvia aerial parts, preferably leaves, the extraction is done with a composition of an organic solvent, i.e. acetone, and activated carbon.

Several methods are known in the art for the extraction of plant parts. One such method is solvent extraction, a method to separate compounds based on their relative solubilities in two different immiscible liquids, usually water (polar) and an organic solvent (non-polar). There is a net transfer of one or more species from the liquid into another liquid phase, generally from aqueous to organic. The transfer is driven by chemical potential, i.e. once the transfer is complete, the overall system of chemical components that make up the solutes and the solvents are in a more stable configuration (lower free energy). In a preferred embodiment of the composition of the invention the extract of green coffee beans is obtained by an extraction with an alcoholic solvent.

In the context of the present invention, the term “solvent” refers to a substance that dissolves a solute, resulting in a solution. A solvent is usually a liquid but can also be a solid, a gas, or a supercritical fluid.

The term “alcoholic solvent” as used herein refers to solvents comprising an ethyl group linked to an hydroxyl group. Examples of alcohol solvents are, without limitation, ethanol, ethylene glycol, isopropanol, methanol, isobutanol, diethylene glycol, etc. In a preferred embodiment of the composition of the invention the green coffee beans extraction is carried out with ethanol, preferably at a ratio of 75% to 25% solvent to water.

The term “activated carbon”, also called activated charcoal, as used herein refers to a form of carbon commonly used to filter contaminants from water and air, among many other uses. It is processed to have small, low-volume pores that increase the surface area available for adsorption or chemical reactions. In a preferred embodiment of the composition of the invention the extraction of the Salvia aerial parts, preferably leaves, is done with a concentration of activate carbon of about 1% w/w to about 15% w/w, preferably about 3.5% w/w, about 4% w/w, about 5% w/w, about 10% w/w.

In order to obtain better results in the extraction process of the Salvia aerial parts, preferably leaves, in a preferred embodiment of the composition of the invention the extraction of the Salvia aerial parts, preferably leaves, is carried out at a temperature of about 50° C. to about 60° C., preferably 56.4° C., for about 1 hour to about 3 hours, preferably about 2 hours, under stirring.

Once the extraction step has been carried out and the resulting plant residues removed, the extract may be further purified by methods already known in the art to achieve higher concentrations of the actives such as the methods described in WO96/34534, which is incorporated here by reference.

Another possible component of the composition of the invention is rosmarinic acid. In a preferred embodiment of the composition of the invention the ratio of chlorogenic acid to the one or more additional component of combination of components is of between 50:1 to 1:50, such as between 25:1 to 1:25, such as between 10:1 to 1:10, such as between 5:1 to 1:5. In a more preferred embodiment of the composition of the invention the ratio of chlorogenic acid to rosmarinic acid is of between 50:1 to about 1:50, such as between about 25:1 to about 1:25, such as between about 10:1 to about 1:10, such as between about 6:1 to about 1:6, preferably between about 5:1 to about 1:5.

In a preferred embodiment of the method of the invention, the concentration of the rosmarinic acid is of at least 0.1% w/w, preferably of at least about 0.5% w/w, more preferably at least about 1% w/w. The term “rosmarinic acid” as used herein refers to a polyphenol constituent of many culinary herbs, including rosemary (Salvia rosmarinus), lemon balm (Melissa officinalis L.), perilla (Perilla frutescens L.), sage (Salvia officinalis L.), mint (Mentha arvense L.), and basil (Ocimum basilicum L.). Rosmarinic acid is an ester formed via the esterification of caffeic acid with 3,4-dihydroxyphenyl lactic acid.

Rosmarinic acid is defined in the entry of the database CAS Registry with the number 20283-92-5 as well as structurally by formula (vii)

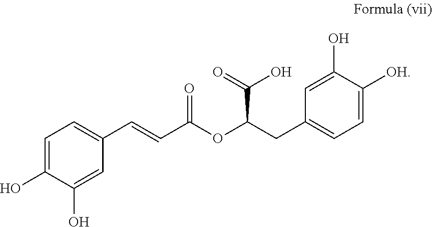 [SMILES: O=C(/C=C/c1ccc(O)c(O)c1)O[C@H](Cc1ccc(O)c(O)c1)C(=O)O]

Rosmarinic acid can be obtained from extract of rosemary aerial parts or an extract of Melissa officinalis aerial parts. In a preferred embodiment of the composition of the invention the rosmarinic acid is provided as an extract of rosemary leaves or an extract of Melissa officinalis aerial parts. In another preferred embodiment of the composition of the invention the rosemary leaves are from the plant species Salvia rosmarinus.

In a preferred embodiment the extract of rosemary leaves is obtained by an extraction with water or an alcoholic solvent. The term “alcoholic solvent” was previously defined and said definition is equally appliable to the current embodiment. In a preferred embodiment of the composition of the invention the rosemary leaves extraction is carried out with water.

Water extractions are well-known in the art and their application is part of the general knowledge of the expert. In a preferred embodiment of the composition of the invention the extraction of the rosemary leaves is carried out at a temperature of about 80° C. to about 100° C., preferably 90° C., for about 1 hour to about 3 hours, preferably about 1.5 hours, by percolation.

In a preferred embodiment of the composition of the invention the extract of rosemary leaves obtained is further filtered and concentrated, preferably concentrated by membrane filtration.

Once the concentration step has been carried out, the extract is purified on polymeric adsorbent resin and the purified extract is spray dried on maltodextrin or formulated on ethanol or propylene glycol.

As previously stated, rosmarinic acid can be obtained from an extract of aerial parts of Melissa officinalis. The term “aerial parts” as used herein refers to, but is not limited to, the shoots, leaves, stems, flowers, fruits and seeds, preferably the leaves or seeds of Melissa officinalis. In a preferred embodiment of the composition of the invention the extract of Melissa officinalis aerial parts is obtained by an extraction with ethanol and activated carbon. In another preferred embodiment the of the composition of the invention the extract of Melissa officinalis aerial parts is obtained with ethanol and activated carbon wherein the ethanol is used at a concentration of about 70% v/v solvent to water and the activated carbon is used at a concentration of about 1% w/v to about 15% w/v, preferably about 3% w/v, about 4% w/v, about 5% w/v, about 6% w/v, about 7% w/v, about 8% w/v, about 10% w/v.

In a preferred embodiment the composition of the invention further comprises caffeine at a ratio of chlorogenic acid to caffeine of between about 15:1 to about 1:15, such as between 10.1 to about 1.10, such as about 1.5:1 to about 10:1, such as about 1.5.1 to about 1:1.5.

In a preferred embodiment the composition of the invention further comprises caffeine at a concentration of between about 0.01% w/w to about 15% w/w, about 0.25% w/v to 12% w/v, about 0.5% w/v to 10% w/v, about 1% w/v to 9% w/v, about 2% w/v to 8% w/v, about 2% w/v to 7% w/v, about 3% w/v to 6% w/v, about 4% w/v to 5% w/v. In a preferred embodiment the composition of the invention further comprises caffeine at a concentration of between about 0.01% w/w to about 1.5% w/v, about 0.25% w/v to 1.5% w/v, about 0.3% w/v to 1.45% w/v, about 0.35% w/v to 1.4% w/v, about 0.4% w/v to 1.35% w/v, about 0.45% w/v to 1.3% w/v, about 0.5% w/v to 1.25% w/v, about 0.55% w/v to 1.2% w/v, about 0.6% w/v to 1.15% w/v, about 0.65% w/v to 1.1% w/v, about 0.7% w/v to 1.05% w/v, about 0.75% w/v to 1% w/v, about 0.8% w/v to 1% w/v, about 0.85% w/v to 1% w/v, about 0.9% w/v to 1% w/v.

The term “caffeine” as used herein refers to the bitter, white crystalline purine, a methylxanthine alkaloid which is defined in the entry of the database CAS Registry with the number 58-08-2 as well as structurally by formula (iv)

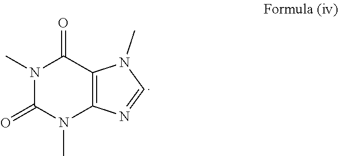

Since the coffee bean is a source for both chlorogenic acid and caffeine, the preparation of the compositions of the invention starting from coffee beans will lead to compositions containing both chlorogenic acid and caffeine. However, for the purposes of the present invention, any plant that comprises chlorogenic acid and/or caffeine may be used, such as yerba mate (Ilex paraguariensis), white tea (Camellia sinensis), winter's bark (Drimys winteri), green tea (Camellia sinensis), elderflower (Sambucus nigra), and Boehmeria caudate. In a preferred embodiment of the composition of the invention the chlorogenic acid and the caffeine are provided as an extract of green coffee beans. The expression “green coffee beans” as used herein refers to unroasted coffee beans. In another preferred embodiment of the composition of the invention the coffee beans are from the plant species Coffea Arabica or Coffea robusta.

Methods to obtain chlorogenic acid and caffeine of coffee beans are widely known in the art and examples are provided in the present description (see Examples).

In a preferred embodiment of the composition of the invention the extract of green coffee beans obtained is further filtered and concentrated. In another preferred embodiment the concentrated extract is supplemented with an organic strong or weak acid.

The term “organic acids” as used herein refers to an organic compound with acidic properties. The most common organic acids are the carboxylic acids, whose acidity is associated with their carboxyl group —COOH. Sulfonic acids, containing the group —SO2OH, are relatively stronger acids. Alcohols, with —OH, can act as acids but they are usually very weak. The relative stability of the conjugate base of the acid determines its acidity. Other groups can also confer acidity, usually weakly: the thiol group —SH, the enol group, and the phenol group. In biological systems, organic compounds containing these groups are generally referred to as organic acids. Examples of organic acids, without limitation are, lactic acid, acetic acid, formic acid, citric acid, oxalic acid, uric acid, malic acid and tartaric acid.

In a preferred embodiment of the composition of the invention the concentration of phenolic diterpene carnosic acid is of between about 0.1% w/v to 5% w/v, about 0.25% w/v to 4.75% w/v, about 0.5% w/v to 4.5% w/v, about 0.75% w/v to 4.25% w/v, about 1% w/v to 4% w/v, about 1.25% w/v to 3.75% w/v, about 1.5% w/v to 3.5% w/v, about 1.75% w/v to 3.25% w/v, about 2% w/v to 3% w/v, about 2.25% w/v to 3% w/v, about 2.5% w/v to 3% w/v.

The term “carnosic acid” in the context of the present description refers to benzenediol abietane diterpene which is found in Salvia rosmarinus, Salvia officinalis, and the like. Carnosic acid is defined in the CAS Registry database with accession number 3650-09-7 as well as structurally by formula (v)

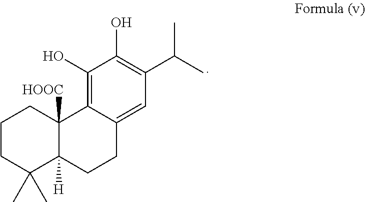 [SMILES: CC(C)c1cc2c(c(O)c1O)[C@@]1(C(=O)O)CCCC(C)(C)[C@@H]1CC2]

In a preferred embodiment of the composition of the invention the ratio of the chlorogenic acid to the phenolic diterpene carnosol acid is of between about 50:1 to 1:50, such as between about 25:1 to about 1:25, such as between about 10:1 to about 1:10, such as between about 5:1 to about 1:5. In a more preferred embodiment of the composition of the invention the ratio of chlorogenic acid to rosmarinic acid is of between about 6:1 to about 1:6, preferably between about 5:1 to about 1:5. In a preferred embodiment the composition of the invention the phenolic diterpene carnosol concentration is of between about 0.001% w/v to about 5% w/v, about 0.01% w/v to 5.0% w/v, about 0.1% w/v to 5.0% w/v, about 0.2% w/v to 4.8% w/v, about 0.3% w/v to 4.7% w/v, about 0.4% w/v to 4.6% w/v, about 0.5% w/v to 4.5% w/v, about 0.6% w/v to 4.4% w/v, about 0.7% w/v to 4.3% w/v, about 0.8% w/v to 4.2% w/v, about 0.9% w/v to 4.1% w/v, about 1% w/v to 4% w/v, about 1.1% w/v to 3.9% w/v, about 1.2% w/v to 3.8% w/v, about 1.3% w/v to 3.7% w/v, about 1.4% w/v to 3.6% w/v, about 1.5% w/v to 3.5% w/v, about 1.6% w/v to 3.4% w/v, about 1.7% w/v to 3.3% w/v, about 1.8% w/v to 3.2% w/v, about 1.9% w/v to 3.1% w/v, about 2% w/v to 3% w/v, about 2.1% w/v to 3% w/v, about 2.2% w/v to 3% w/v, about 2.3% w/v to 3% w/v, about 2.4% w/v to 3% w/v, about 2.5% w/v to 3% w/v

The term “carnosol”, such as the carnosic acid, as used herein refers to the phenolic diterpene found in in Salvia rosmarinus, Salvia officinalis, and the like. Carnosol is defined in the CAS Registry database with accession number 5957-80-2 as well as structurally by formula (vi)

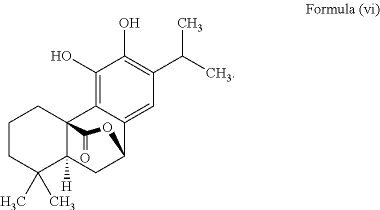

Carnosic acid and carnosol can be obtained from extract of rosemary leaves. In a preferred embodiment of the composition of the invention the phenolic diterpene carnosic acid and/or the carnosol as the case may be is provided as an extract of rosemary leaves. In another preferred embodiment of the composition of the invention the rosemary leaves are from the plant species Salvia rosmarinus.

Several procedures to obtain the extract of rosemary aerial parts are available in the literature, such as in the document WO96/34534, which is incorporated here by reference. In a preferred embodiment of the composition of the invention the extract of rosemary aerial parts is obtained by a method comprising the steps of: (i) extracting the aerial parts with an organic solvent and, optionally (ii) further purifying the extract.

The term “organic solvents” as used herein refers to carbon-based substances capable of dissolving or dispersing one or more other substances. Examples of organic solvents, without limitation, are acetic acid, acetone, acetonitrile, benzene, 1-butanol, 2-butanol, chloroform, ethanol, glycerin, etc. In a preferred embodiment of the composition of the invention the organic solvent used in the extract of rosemary leaves is acetone.

Once the extraction step has been carried out and the resulting plant residues removed, the extract may be further purified by methods already known in the art to achieve higher concentrations of the actives such as the methods described in WO96/34534, which is incorporated here by reference.

Once obtained, the extracts herein mentioned which are used in the composition of the invention may be filtered, dried, further purified, concentrated, etc. Techniques for applying such processes to extracts are well known in the art and the expert would be aware of such methods and be able to select the best parameters to perform such techniques. In a preferred embodiment of the composition of the extract is processed by a technique selected from the filtering, drying, concentrating, milling, filtering and drying, filtering and concentrating, filtering and concentrating and drying, filtering and drying and milling or concentrating and drying and milling. In another preferred embodiment of the composition of the invention the extract is dried by vacuum oven at a temperature of about 50° C. to about 100° C., preferably about 75° C., more preferably about 85° C.

The extracts herein mentioned can further be formulated in a carrier, either liquid or solid. The extract obtained in order to form the synergistic composition of the invention can further comprise a suitable carrier. In a preferred embodiment the composition of the invention further comprises a carrier. The term “carrier” as used herein refers to any type of material compatible with the intended use, on which a molecule, a compound, a substance (such as a plant extract) or a composition can be supported, either by physical interaction or by chemical interaction with the carrier material. As an example of the interaction of the carrier material with a plant extract or a composition, the interaction has to not prevent the plant extract or composition to exert its desirable effect, i.e., reduce its antioxidant activity. In a preferred embodiment of the composition of the invention the carrier is liquid or solid. In another preferred embodiment of the composition of the invention the carrier is selected from the group consisting of: ethanol, maltodextrin and monopropylene glycol (MPG).

### Processes and Uses of the Invention

As previously mentioned substances with antioxidant properties find several uses, in particular in the agro-alimentary industry. Therefore an aspect of the present invention relates to the use of the composition of the invention as an antioxidant, from here onwards the use I of the invention.

Another aspect of the present invention relates to a process preventing and/or inhibiting oxidation of a foodstuff, the process comprising the steps of contacting the foodstuff with the composition of the invention, from here onwards the process of the invention.

A further aspect of the present invention relates to the use of the composition of the invention for preventing and/or inhibiting oxidation of a foodstuff, from here onwards the use II of the invention.

All previous embodiments and definitions described in relation with the previous aspects are equally valid for the current aspect and its embodiments.

Thus in certain embodiments, the present invention relates to a process preventing and/or inhibiting oxidation of a food or beverage product for humans or animals, a nutritional supplement, a nutraceutical formulation, a fragrance or flavouring, a pharmaceutical or veterinary formulation, an oenological or cosmetic formulation, the process comprising the steps of contacting the food or beverage product for humans or animals, nutritional supplement, nutraceutical formulation, fragrance or flavouring, pharmaceutical or veterinary formulation, oenological or cosmetic formulation with a composition comprising a chlorogenic acid and one or more additional component or combination of components selected from a group consisting of:
(i) rosmarinic acid; and
(ii) phenolic diterpenes,
   wherein the ratio of chlorogenic acid to the one or more additional component or combination of components is of between about 100:1 to about 1:100.

Thus in certain embodiments, the present invention relates to the use of a composition comprising a chlorogenic acid and one or more additional component or combination of components selected from a group consisting of:
(i) rosmarinic acid; and
(ii) phenolic diterpenes,
   wherein the ratio of chlorogenic acid to the one or more additional component or combination of components is of between about 100:1 to about 1:100, as an antioxidant.

Thus in certain embodiments, the present invention relates to a process for preventing and/or inhibiting oxidation of a food or beverage product for humans or animals, a nutritional supplement, a nutraceutical formulation, a fragrance or flavouring, a pharmaceutical or veterinary formulation, an oenological or cosmetic formulation, the process comprising the steps of contacting the food or beverage product for humans or animals, the nutritional supplement, the nutraceutical formulation, the fragrance or flavouring, the pharmaceutical or veterinary formulation, the oenological or the cosmetic formulation with a composition comprising a chlorogenic acid and one or more additional component or combination of components selected from a group consisting of:
(i) rosmarinic acid; and
(ii) phenolic diterpenes,
   wherein the ratio of chlorogenic acid to the one or more additional component or combination of components is of between about 100:1 to about 1:100.

In certain embodiments of the uses and processes of the invention, the ratio of chlorogenic acid to the one or more additional components or combination of components is of about 95:5, 97:7, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 7:97, about 5:95. In certain embodiments, the ratio of chlorogenic acid to the one or more additional components or combination of components is of between 50:1 to 1:50, preferably between 25:1 to 1:25. In certain embodiments, the ratio of chlorogenic acid to the one or more additional components or combination of components is of between about 50:1 to about 1:50, such as between about 25:1 to about 1:25, such as between about 10:1 to about 1:10, such as between about 5:1 to about 1:5.

In certain embodiments of the uses and processes of the invention, the ratio of chlorogenic acid to rosmarinic acid is of between about 50:1 to about 1:50, such as between about 25:1 to about 1:25, such as between about 10:1 to about 1:10, such as between about 6:1 to about 1:6, preferably between about 5:1 to about 1:5.

In certain embodiments of the uses and processes of the invention, the ratio of the chlorogenic acid to the phenolic diterpene carnosol acid is of between about 50:1 to 1:50, such as between about 25:1 to about 1:25, such as between about 10:1 to about 1:10, such as between about 5:1 to about 1:5. In certain embodiments, the ratio of chlorogenic acid to rosmarinic acid is of between about 6:1 to about 1:6, preferably between about 5:1 to about 1:5. In certain embodiments, the phenolic diterpene carnosol concentration is of between about 0.001% w/v to about 5% w/v, about 0.01% w/v to 5.0% w/v, about 0.1% w/v to 5.0% w/v, about 0.2% w/v to 4.8% w/v, about 0.3% w/v to 4.7% w/v, about 0.4% w/v to 4.6% w/v, about 0.5% w/v to 4.5% w/v, about 0.6% w/v to 4.4% w/v, about 0.7% w/v to 4.3% w/v, about 0.8% w/v to 4.2% w/v, about 0.9% w/v to 4.1% w/v, about 1% w/v to 4% w/v, about 1.1% w/v to 3.9% w/v, about 1.2% w/v to 3.8% w/v, about 1.3% w/v to 3.7% w/v, about 1.4% w/v to 3.6% w/v, about 1.5% w/v to 3.5% w/v, about 1.6% w/v to 3.4% w/v, about 1.7% w/v to 3.3% w/v, about 1.8% w/v to 3.2% w/v, about 1.9% w/v to 3.1% w/v, about 2% w/v to 3% w/v, about 2.1% w/v to 3% w/v, about 2.2% w/v to 3% w/v, about 2.3% w/v to 3% w/v, about 2.4% w/v to 3% w/v, about 2.5% w/v to 3% w/v

As used herein, the term “oxidation” refers to the exposure of the composition and/or foodstuff to conditions (such as for example an amount of atmospheric oxygen) that is sufficient enough to significantly oxidize it. The oxidation will occur via reactions including but not limited to reactions promoted by oxygen or induced by the presence of metal, such as iron (reaction of Fenton). As used herein, the expression “preventing and/or inhibiting oxidation of a foodstuff” refers to the reduction of the oxidation which occurs to the foodstuff in the presence of the composition of the invention in comparison to the oxidation which occurs in the absence of said composition. The term “prevention” refers to the complete absence of oxidation in the foodstuff when the foodstuff comprises the composition of the invention. The term “inhibiting” refers to the reduction of the level of oxidation which occurs to the foodstuff when in the presence of oxygen. In a particular embodiment of the process of the invention the oxidation of the foodstuff is inhibited by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, in comparison with the level of oxidation of the foodstuff without the composition of the invention. Methods for measuring the food oxidation are well known in the art and in the literature, e.g., Barriuso et al, 2013 (European Food Research and Technology volume 236, pages 1-15).

In a particular embodiment, the process of the invention or the uses I or II of the invention is in the manufacture of a agro-alimentary product.

The term “agro-alimentary product” as used herein refers to products which are used on making, processing and preparing food products for animal consumption, preferably human consumption.

In a particular embodiment, the process of the invention or the uses I or II of the invention is in the manufacture of foodstuff.

The term “foodstuff” as used herein is intended to mean a substance which is suitable for human or animal consumption. In addition, the term foodstuff as used herein also is meant to include beverages and food materials which are used in the preparation of a foodstuff. As used herein the term foodstuff includes, without limitation, beverages, eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions, sauces, beverages, patisseries, oils, bakery, filling and edible ink.

In a preferred embodiment of the process of the invention or the uses I or II of the invention the foodstuff is a product selected from a group consisting of: eggs, mayonnaise, salad dressings, sauces, ice creams, egg powder, modified egg yolk, breads, cakes, sweet dough products, laminated doughs, liquid batters, muffins, doughnuts, biscuits, crackers, cookies, chocolate, candies, caramels, halawa, gums, bubble gum, soft bubble gum, chewing gum, puddings, sorbets, ice cream, ice milk, cheese, butter, milk, coffee cream, whipped cream, custard cream, milk drinks, yoghurts, mousses, whipped vegetable creams, meat, processed meat products, oils, edible oils, fats, oil-in-water emulsions, water-in-oil emulsions, margarine, mayonnaise, dips, cream based sauces, cream based soups, beverages, spice emulsions, sauces, patisseries, dairy products, oils, bakery, filling and edible ink.

In a further preferred embodiment of the process of the invention or the uses I or II of the invention the foodstuff is a meat product. The term “meat product” is used herein with the meaning of those products extracted from poultry, fish, cattle, pork, sheep, etc., both with and without fat, either in the shape of meat cut or shred, ground meat, frozen meat, semi-frozen meat or refrigerated meat, either processed or not processed. In a further preferred embodiment of the process of the invention or the uses I or II of the invention the foodstuff is a pork meat product.

In a further preferred embodiment of the process of the invention or the uses I or II of the invention the foodstuff is a beverage, preferably a lemon flavoured beverage being the flavour natural and/or artificial.

The content of the compositions of the invention for the manufacture of the foodstuffs will be dependent on the type of foodstuff as well as the intended use of such product.

### Foodstuff, Nutraceutical or Dietary Supplement and Other Products of the Invention

The use of the invention allows the manufacture of a food or beverage product for humans or animals, a nutritional supplement, a nutraceutical formulation, a fragrance or flavouring, a pharmaceutical or veterinary formulation, an oenological or cosmetic formulation with enhanced shelf-life (i.e reduces oxidation of said product). Therefore, another aspect of the present invention relates to a product (such as a food or beverage product for humans or animals, a nutritional supplement, a nutraceutical formulation, a fragrance or flavouring, a pharmaceutical or veterinary formulation, an oenological or cosmetic formulation) comprising the composition of the invention, from here onwards the foodstuff of the invention, or a nutraceutical or dietary supplement of the invention, etc, comprising the composition of the invention and a food or dietary acceptable adjuvant.

All previous embodiments and definitions described in relation with the previous aspects are equally valid for the current aspect and its embodiments.

The term “nutraceutical” as used herein refers to substances useful in both nutritional and pharmaceutical fields of application. Thus, nutraceutical compositions can be used as supplements to food and beverages and as pharmaceutical formulations for enteral or parenteral application which may be solid formulations, such as capsules or tablets, or liquid formulations, such as solutions or suspensions.

The expression “food or dietary acceptable adjuvant” as used herein refers to any food material that can be used together with the main foodstuff material in order to enhance, preserve, and improve the characteristics of the foodstuff main material. Adjuvants can also serve as fillers, diluents, viscosity regulators, etc.

In a preferred embodiment the foodstuff of the invention the foodstuff is a meat product. In another preferred embodiment of the foodstuff of the invention, the foodstuff is a pork meat product.

The expert will know how to adjust the quantity of the compositions in the foodstuff. In a preferred embodiment of the products of the invention (such as a food or beverage product for humans or animals, a nutritional supplement, a nutraceutical formulation, a fragrance or flavouring, a pharmaceutical or veterinary formulation, an oenological or cosmetic formulation), the composition in the product (such as a foodstuff, etc) comprises a ratio of chlorogenic acid to phenolic diterpenes of between about 0.1:1 to about 1:0.1, preferably between about 0.8:1 to about 1:0.8.

In another preferred embodiment of the product (such as a foodstuff, etc) of the invention, the composition in the product (such as a foodstuff, etc) comprises between:
about 0.1% w/w to about 55.0% w/w, preferably between about 2.0% w/w to of 25% w/w of chlorogenic acid;
about 0.1% w/w to about 10.0 w/w, preferably between 0.2% w/w to about 5% w/w of caffeine; and
at least about 0.1% w/w of phenolic diterpenes.

In another preferred embodiment of the product (such as a foodstuff, etc) of the invention the composition in the product (such as a foodstuff, etc) comprises between about 2.25% w/w to about 2.4% w/w chlorogenic acid, between about 0.25% w/w to about 1.5% w/w caffeine and more than 2.5% w/w phenolic diterpenes.

In another preferred embodiment of the product (such as a foodstuff, etc) of the invention the concentration in the product (such as a foodstuff, etc) is of about 0.5 parts per million (ppm) to about 1000 ppm, preferably about 400 ppm to about 600 ppm.

In another preferred embodiment, the product (such as a foodstuff, etc) of the invention comprises between about 1 ppm to about 1000 ppm of chlorogenic acid, such as from about 5 ppm, 10 ppm, 20 ppm, 30 ppm, 40 ppm, 50 ppm, 60 ppm, 70 ppm, 80 ppm, 90 ppm, 100 ppm, 200 ppm, 300 ppm, 400 ppm, 500 ppm, 600 ppm, 700 ppm, 800 ppm, 900 ppm to about 1000 ppm, 900 ppm, 800 ppm, 700 ppm, 600 ppm, 500 ppm, 400 ppm, 300 ppm, 200 ppm, 100 ppm, 90 ppm, 80 ppm, 70 ppm, 60 ppm, 50 ppm, 40 ppm, 30 ppm, 20 ppm, 10 ppm, or to 5 ppm. In certain embodiments, the product (such as a foodstuff, etc) comprises from about 50 ppm of chlorogenic acid, preferably about 1 ppm to about 40 ppm of chlorogenic acid, more preferably about 1 ppm to about 30 ppm of chlorogenic acid, even more preferably about 9 ppm to about 13.5 ppm of chlorogenic acid in respect to the final weight of the product.

In another preferred embodiment, the product (such as a foodstuff, etc) of the invention, comprises between about 1 ppm to about 1000 ppm of phenolic diterpenes, such as from about 5 ppm, 10 ppm, 20 ppm, 30 ppm, 40 ppm, 50 ppm, 60 ppm, 70 ppm, 80 ppm, 90 ppm, 100 ppm, 200 ppm, 300 ppm, 400 ppm, 500 ppm, 600 ppm, 700 ppm, 800 ppm, 900 ppm to about 1000 ppm, 900 ppm, 800 ppm, 700 ppm, 600 ppm, 500 ppm, 400 ppm, 300 ppm, 200 ppm, 100 ppm, 90 ppm, 80 ppm, 70 ppm, 60 ppm, 50 ppm, 40 ppm, 30 ppm, 20 ppm, 10 ppm, or to 5 ppm. In certain embodiments, the product comprises from about 50 ppm of phenolic diterpenes; preferably 1 ppm to about 50 ppm phenolic diterpenes, preferably about 1 ppm to about 40 ppm phenolic diterpenes, more preferably about 1 ppm to about 30 ppm phenolic diterpenes, even more preferably about 10 ppm to about 15 ppm in respect to the final weight of the product.

In another preferred embodiment, the product (such as a foodstuff, etc) of the invention comprises between about 1 ppm to about 50 ppm phenolic diterpenes, preferably about 1 ppm to about 40 ppm phenolic diterpenes, more preferably about 1 ppm to about 30 ppm phenolic diterpenes, even more preferably about 10 ppm to about 15 ppm in respect to the final weight of the product.

In another preferred embodiment, the product (such as a foodstuff, etc) of the invention is a meat product and the concentration of the composition in the foodstuff is of between about 5 ppm to about 20 ppm of chlorogenic acid and of between about 5 ppm to about 25 ppm of phenolic diterpenes in respect to the final weight of the product.

In another preferred embodiment of the product (such as a foodstuff, etc) of the invention is a meat product and the concentration of the composition in the foodstuff is of about 13.5 ppm of chlorogenic acid and of about 15 ppm of phenolic diterpenes in respect to the final weight of the product.

In another preferred embodiment of the product (such as a foodstuff, etc) of the invention is a pork meat product and the concentration of the composition in the foodstuff is of about 9 ppm of chlorogenic acid and of about 10 ppm of phenolic diterpenes in respect to the final weight of the product.

In another embodiment of the foodstuff of the invention the foodstuff is a meat product and the concentration of the composition in the foodstuff is of about 13.5 ppm of chlorogenic acid and of about 15 ppm of phenolic diterpenes.

In another embodiment of the foodstuff of the invention the foodstuff is a meat product and wherein the concentration of the composition in the foodstuff is from about 5 ppm to about 15 ppm, such as about 9 ppm of chlorogenic acid and from about 5 ppm to about 20 ppm of phenolic diterpenes, such as of about 10 ppm or about 15 ppm of phenolic diterpenes.

In another preferred embodiment of the foodstuff of the invention is a pork meat product and the concentration of the composition in the foodstuff is of about 9 ppm of chlorogenic acid and of about 10 ppm of phenolic diterpenes in respect to the final weight of the product.

In another preferred embodiment, the product (such as a foodstuff, etc) of the invention, comprises between about 1 ppm to about 1000 ppm of rosmarinic acid, such as from about 5 ppm, 10 ppm, 20 ppm, 30 ppm, 40 ppm, 50 ppm, 60 ppm, 70 ppm, 80 ppm, 90 ppm, 100 ppm, 200 ppm, 300 ppm, 400 ppm, 500 ppm, 600 ppm, 700 ppm, 800 ppm, 900 ppm to about 1000 ppm, 900 ppm, 800 ppm, 700 ppm, 600 ppm, 500 ppm, 400 ppm, 300 ppm, 200 ppm, 100 ppm, 90 ppm, 80 ppm, 70 ppm, 60 ppm, 50 ppm, 40 ppm, 30 ppm, 20 ppm, 10 ppm, or to 5 ppm. In certain embodiments, the product comprises from about 50 ppm of rosmarinic acid; preferably about 1 ppm to about 50 ppm of rosmarinic acid, preferably about 1 ppm to about 40 ppm rosmarinic acid, more preferably about 1 ppm to about 30 ppm of rosmarinic acid, even more preferably about 10 ppm to about 15 ppm of rosmarinic acid in respect to the final weight of the product.

In another embodiment, the product (such as a foodstuff, etc) of the invention comprises between about 1 ppm to about 50 ppm rosmarinic acid, preferably about 1 ppm to about 40 ppm rosmarinic acid, more preferably about 1 ppm to about 30 ppm rosmarinic acid, even more preferably about 10 ppm to about 15 ppm in respect to the final weight of the product.

In another preferred embodiment, the product (such as a foodstuff, etc) of the invention is a beverage product and the concentration of the composition in the foodstuff is of between about 5 ppm to about 20 ppm of chlorogenic acid and of between about 5 ppm to about 25 ppm of rosmarinic acid in respect to the final weight of the product.

In another preferred embodiment, the product (such as a foodstuff, etc) of the invention is a beverage product and the concentration of the composition in the foodstuff is of about 13.5 ppm of chlorogenic acid and of about 15 ppm of rosmarinic acid in respect to the final weight of the product.

In another preferred embodiment, the product (such as a foodstuff, etc) of the invention is a beverage product and the concentration of the composition in the foodstuff is of about 9 ppm of chlorogenic acid and of about 10 ppm of rosmarinic acid. in respect to the final weight of the product

In another preferred embodiment, the foodstuff of the invention is a beverage product and the concentration of the composition in the foodstuff is of about 9 ppm of chlorogenic acid and of about 10 ppm of rosmarinic acid in respect to the final weight of the product.

In another preferred embodiment, the foodstuff of the invention is a beverage product and the concentration of the composition in the foodstuff is of about 9 ppm of chlorogenic acid and of about 1.5 ppm of rosmarinic acid in respect to the final weight of the product.

In another preferred embodiment the foodstuff of the invention is a beverage. The term “beverage” as used herein refers to any potable or edible liquid. In another preferred embodiment of the foodstuff the beverage is a lemon flavoured beverage, being the flavour natural and/or artificial.

The expert will know how to adjust the quantity of the compositions in the beverage. In another preferred embodiment of the foodstuff of the invention the composition comprises between about 4% to about 5% chlorogenic acid, between about 0.3% to about 3% caffeine and between about 0.5% to about 1% rosmarinic acid.

In a preferred embodiment of the foodstuff of the invention, the composition is present in the beverage at a concentration of about 50 to 500 ppm, preferably about 100 ppm to about 300 ppm.

In another preferred embodiment of the foodstuff of the invention, the foodstuff is a beverage, preferably a lemon flavoured beverage, wherein the concentration of the composition in the foodstuff is of between about 1 ppm to about 500 ppm of chlorogenic acid, such as from 200 ppm to 500 ppm of chlorogenic acid, such as 400 ppm of chlorogenic acid and of between about 0.5 ppm to about 50 ppm of rosmarinic acid, such as from 20 to 40 ppm, such as 30 ppm rosmarinic acid.

In another preferred embodiment of the foodstuff of the invention, the foodstuff is a beverage, preferably a lemon flavoured beverage, wherein the concentration of the composition in the foodstuff is of about 9 ppm of chlorogenic acid and of about 1.5 ppm of rosmarinic acid in respect to the final weight of the product.

The invention will be described by way of the following examples which are to be considered as merely illustrative and not limitative of the scope of the invention.

### EXAMPLES

### Example 1: Products Description

TABLE 1
Summary of the extract products obtained

| | | Plant and | | | |
|---|---|---|---|---|---|
| Extract | | part of | | | Active |
| number | Name | plant | Process | Composition | contents |
| A | Rosemary | Rosemary | Acetone + pH | 50-60% w/w PG*, | carnosic acid: |
| | CA WS | leaves | modification | 30-40% w/w P80, | 5-5.5% w/w |
| | | | to purify | 5-15% w/w Nat | carnosic acid + |
| | | | extract | Extract | carnosol: 5.5- |
| | | | | | 6.5% w/w |
| B | GBC liq | Green | EtOH 75% | 50-60% w/w Nat | chlorogenic acid: |
| | | coffee | | extract, 20-25% | 4.5-4.8% w/w |
| | | beans | | MPG, 3-8% w/w | caffeine: |
| | | | | citric acid and | 0.5-3% w/w |
| | | | | 12-22% w/w | |
| | | | | water | |
| C | GBC (pwd) | Green | EtOH 75% | 25% w/w | chlorogenic acid: |
| | | coffee | | extract and | 4.5-4.8% w/w |
| | | beans | | 75% w/w | caffeine: |
| | | | | maltodextrin | 0.5-3% w/w |
| D | Sage pwd | Salvia | Acetone | 100% w/w Nat | phenolic |
| | | leaves | | extract | diterpenes > |
| | | | | | 50% w/w |
| E | Sage WS | Salvia | Acetone | PG 45-60% w/w | phenolic |
| | | leaves | | P 80: 30-40% w/w | diterpenes > |
| | | | | Nat extract: | 5% w/w |
| | | | | 7-18% w/w | |
| F | Rosemary | Rosemary | Water + | 27% w/w extract | Rosmarinic acid: |
| | RA | leaves | Purification | and | 9-10% w/w |
| | | | | 72% w/w | |
| | | | | maltodextrin | |
| G | Lemon | Lemon | EtOH 70% | 30-70% w/w | Rosmarinic acid |
| | Balm | Balm | | extract | 4-4.5% w/w |
| | | leaves | | 70-30% w/w PG | |

*Nat extract = Natural extract,
P80: Polysorbate 80,
MPG = PG = Propylene glycol,
Malto = maltodextrin

### 1. Rosemary CA WS (Table 1, Extract A)

The detailed procedure to prepare the composition of Rosemary extract was described in the U.S. Pat. No. 5,859,293 (PCT WO96/34534), which is incorporated herein by reference in its entirely. The procedure is briefly summarized as follows: The Rosemary leaves were extracted with acetone at room temperature. After the extraction was completed, the acetone extract was filtered to separate the solution from Rosemary leaves and concentrated under reduced pressure to make concentrated native extract. To the concentrated native extract, aqueous sodium carbonate (Na₂Co₃) was added to dissolve carnosic acid and other organic acids, while base insoluble substances were precipitated out. The solution was filtered to separate from solid, and the filtrate was further concentrated under reduced pressure. Once finishing concentration is achieved, phosphoric acid (H₃PO₄) was added and the acid insoluble substances (including carnosic acid, carnosol, and carnosic derivatives) were precipitated from the concentrated solution. Through filtering, the precipitated solid was subsequently separated from liquid and rinsed with water to remove impurities. Last, the solid was dried in a vacuum oven and then milled into powder.

Then the product can be formulated on different carrier, solid or liquid, to adjust its physical properties (physical forms, solubility, color . . . )

### 2. Green Coffee Beans Extract (Table 1, Extracts B and C)

Green coffee beans of Coffea arabica or of Coffea robusta were ground and extracted using alcoholic extraction. Any other plant or organism comprising chlorogenic acid, or any other process providing or yielding chlorogenic acid, may be used.

Here, the extraction solvent was composed from 75% ethanol and 25% water.

Thus obtained extract was filtered and concentrated. Concentrated extract was supplemented with organic or weak or strong acid, in here with citric acid. The obtained extract was further filtered and concentrated with or without liquid carrier like propylene glycol. To obtain extract C, the concentrated native extract was spray dried on maltodextrin.

### 3. Salvia Extracts (Table 1, Extracts D and E)

Salvia officinalis or Salvia apiana aerial parts (leaves and stems) were extracted using 2-5% activated carbon with acetone at reflux (56.4° C.) for 2 hours under mechanical stirring. After the extraction was completed, the acetone extract was filtered to remove the plant residues. The resulting acetonic extract was concentrated under reduced pressure. Further purification was performed as described in WO 96/34534. The extract D.

Then the product can be formulated on different carrier, solid or liquid (extract E), to adjust its physical properties (physical forms, solubility, color . . . )

### 4. Rosemary RA Extract (Table 1, Extract F)

Rosemary leaves were extracted by percolation with water at 90° C. for 1 h 30. After the extraction was completed, the water extract was concentrated by membrane filtration.

Once the concentration step has been carried out, the extract is purified on polymeric adsorbent resin and the purified extract is spray dried on maltodextrin.

### Example 2. Methods

### 2.1—In Vitro Test ORAC

Determination of the antioxidant capability by Oxygen Radical Absorbance Capacity was conducted on a fluorescence micro plate reader Tecan Infinite, following the procedure of Ou et al. 2013 (AOAC Official Method 2012.23 Total Antioxidant Activity). The method describes how to carry out the analysis for lipophilic and hydrophilic sample.

ORAC-Assay measures the capacity of a sample to bind oxygen radicals. Reactive oxygen species generators (AAPH) are added in parallel reactions that contain equal amounts of a fluorescent probe (Fluorescein sodium salt). Reactions contain either a buffer blank or antioxidant samples and standard (Trolox). The antioxidant capacity of a sample is the net difference between the area under the curve (AUC) of the sample and that of the blank. This net AUC of sample is compared to the net AUC of Trolox standard concentration in order to have the ORAC value of the sample expressed as μmol Trolox equivalent per gram (μmol TE/g).

The result obtained is proportional to the quantity of extract. A blend of products will give an ORAC value proportional on ORAC of each extract weighted by their proportion in the blend

$$O R A C ( blend ) = \sum ( O R A C of extract alone * proportion of extract in the blend )$$

Example: For a blend of 30% extract 1 at ORAC=2000 μmol TE/g and 70% of extract 2 at ORAC=700 μmol TE/g, we expected that the ORAC value of the blend gives:

$$( 2000 \times 0.3 ) + ( 700 \times 0.7 ) = 1090 μmol T E / g$$

### 2.2. In Vitro Test CAT

Determination of the antioxidant capability by Conjugated Autoxidizable Triene was conducted on a micro plate reader Tecan Infinite, following the procedure of Laguerre et al. (Analytical Biochemistry 380 (2008) 282-290).

As ORAC-Assay, CAT-Assay measures the capacity of a sample to bind oxygen radicals. Reactive oxygen species generators (AAPH) are added in parallel reactions that contain equal amounts of an absorbance probe (emulsion). Reactions contain either a buffer blank or antioxidant samples and standard (Trolox). The antioxidant capacity of a sample is the net difference between the area under the curve (AUC) of the sample and that of the blank. This net AUC of sample is compared to the net AUC of Trolox standard concentration in order to have the CAT value of the sample expressed as μmol Trolox equivalent per gram (μmol TE/g).

As ORAC, the result obtained is proportional to the quantity of extract. A blend of products will give a CAT value proportional on CAT of each extract weighted by their proportion in the blend

$$C A T ( blend ) = \sum ( C A T of extract alone * proportion of extract in the blend )$$

### 2.3. Test in Meat Application (Pork)

For each modality, the meat (composition precised in the table 2)

TABLE 2
Composition of pork meat used

| Ingredients | % |
|---|---|
| Pork shoulder meat | 81.00 |
| Pork backfat | 13.00 |
| Salt | 1.80 |
| Pepper | 0.15 |
| Sodium lactate (60%) | 4.20 |

was mixed by hand with the quantity of extract precised in the table 3 until total complete incorporation.

TABLE 3
Dosage extracts in pork application

| Dosage to be tested | | Actives content |
|---|---|---|
| in Pork (ppm) | Codification | in meat (ppm) |
| 0 | No AOX serie 1 | 0 |
| 600 | 600 ppm (50% | DTP: 15 |
| | B + 50% E) | Chloro: 13.5 |
| 0 | No AOX serie 2 | 0 |
| 400 | 400 ppm (50% | DTP: 10 |
| | B + 50% E) | Chloro: 9 |

Patties were formed and stored at 4° C. in dark under modified atmosphere (MAP) during 10 days. Samples were analysed after 3, 6 and 10 days of storage by TBars assay.

TBA-RS (thiobarbituric acid-reactive substances) is a common analysis of oxidation degree in meat and fish products. The 3 carbons compound malonaldehyde (MDA) is a major carbonyl decomposition molecule of autoxidized, polyunsaturated materials of secondary oxidation. The basic principle of the method is the reaction of one molecule of malonaldehyde with two molecules of TBA to form a pink pigment malonaldehyde-TBA complex, which can be quantified spetrophotometrically at 509, 532 and 580 nm.

For each modality, an amount of 150 g of minced pork was mixed with the adequate level of extract, by hand, until complete incorporation. A patty was formed and cooked in the oven (170° C.) until the temperature at core reaches 70° C.

The patties were stored at 4° C. in a lightbox without any packaging (simply put on a plate to maximize oxygen contact to accelerate oxidation process).

The TBars measurements were done as described previously in Zhang et al, Food Science and Human Wellness, 5, 39-48 (2016))

Two series have been launched, at different time and with different batch of meat, with the blend sage/green bean coffee.

### 2.4. Test in Beverages

Lemon flavour+12 ppm of Citral was added in a standard beverage matrix (acidified and sugar water), with or without our antioxidant extracts (dosages in Table 4). Then “aged” samples were subjected to a 4 weeks accelerated aging process at 40° C. and “fresh” samples were stored at 4° C.

Sensory profiling was conducted on “fresh” and “aged” samples with and without antioxidants to understand how well the solutions work. Flavor Impression profiling consists of quantifying the sensory characteristics of products through open group discussion. Specialists trained panelists taste the full product set and generate descriptors specifically for the samples to be assessed. They rate intensity scores for each of these descriptors individually, then discuss as a group what each has rated, and agree on a final consensus profiling. The test is run in a discussion room, and results are collected by the panel leader.

TABLE 4
Dosages of extracts and actives in beverage application

| | Dosage of extract | Dosage of actives |
|---|---|---|
| Name | in beverage (ppm) | (ppm) |
| Green Coffee Bean | 440 ppm | 20 ppm |
| Rosemary | 30 ppm | 3 ppm |
| Rosemary + Green | 15 + 200 | 1.5 + 9 |
| Coffee Bean | ppm | ppm |

### Example 3: Results

### 1. In Vitro ORAC and CAT Antioxidant Tests

### 1. Blend Containing Rosemary Extract and Green Coffee Bean Extract

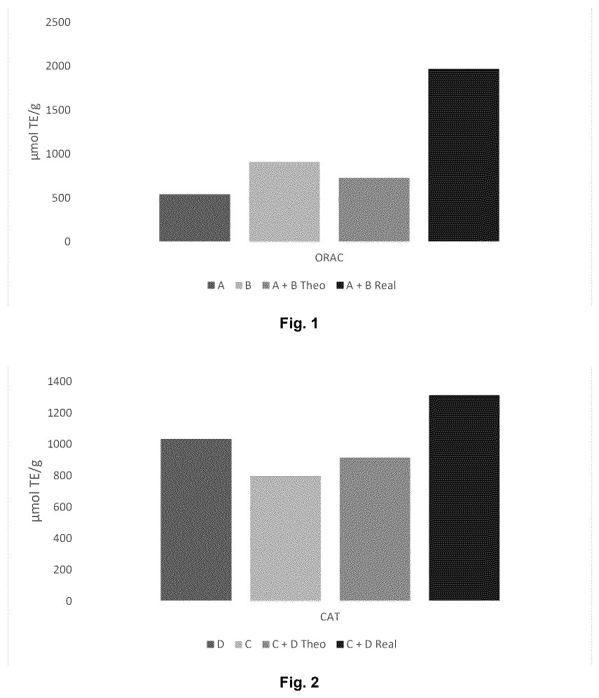

As can be see in table 4 and FIG. 1, when extract A is blended with extract B, the expected ORAC value can be determined as follows:

$$O R A C value blend theoretical = 0.5 \times O R A C ( A ) + 0.5 \times O R A C ( B )$$

$$= 0.5 \times 541.5 + 0.5 \times 913.94$$

$$O R A C value blend theoretical = 727.72$$

After the analysis of the blend containing 50% of extract A and 50% of extract B, the ORAC value was 2.7 times higher than the expected value, indicating a synergy due to the blend of these products.

TABLE 4
ORAC measurements

| Description extrait | ORAC |
|---|---|
| A | 541.5 |
| B | 913.94 |
| A + B (50/50) Theo | 727.72 |
| A + B (50/50) Real | 1966.53 |

2. Blend Sage Extract with Green Coffee Bean Extract

As can be seen in the table 5 and in FIG. 2, when extract C is blended with extract D, an expected CAT value obtained is:

$$C A T value blend theoretical = 0.5 \times C A T ( C ) + 0.5 \times C A T ( D )$$

$$C A T value blend theoretical = 0.5 \times 1031.82 + 0.5 \times 795.7$$

$$C A T value blend theoretical = 913.76$$

After the analysis of the blend containing 50% of extract A and 50% of extract B, the CAT value was 1.4 times higher than expected. This clearly indicates the existence of a synergistic antioxidant effect as a result of the blend of the these products.

TABLE 5
CAT measurements

| Description extrait | CAT |
|---|---|
| D | 1031.82 |
| C | 795.7 |
| C + D Theo | 913.76 |
| C + D Real | 1313.1 |

### 3. Results in Meat Application (Pork)

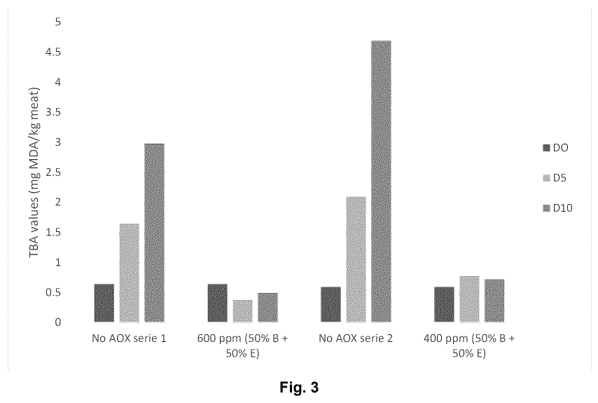

As we can see in FIG. 3 and table 6, a clear increase of TBArs value was observed for the negative controls over time, reaching almost 3 meqMDA/kg for the first series and more than 4.5 meqMDA/kg for the second after 10 days—shelf life of the meat. In comparison, the meat containing the blend of green bean coffee and sage extracts at different dosage stay below 0.8 meqMDA/kg after the shelf life.

The use of natural antioxidant solutions seems to prevent lipid oxidation mechanism.

TABLE 6
TBArs results in pork meat

| | Tbars | | |
|---|---|---|---|
| Codification | D 0 | D 5 | D 10 |
| No AOX serie 1 | 0.64 | 1.65 | 2.98 |
| 600 ppm (50% | 0.64 | 0.38 | 0.49 |
| B + 50% E) | | | |
| No AOX serie 2 | 0.59 | 2.1 | 4.69 |
| 400 ppm (50% | 0.59 | 0.78 | 0.72 |
| B + 50% E) | | | |

### 4. Results in Beverage Applications

Sensory profiling was conducted on fresh and aged samples with and without antioxidants to understand how well the solutions work.

As we can see in the Table 7, Rosemary alone at 30 ppm and Green Bean Coffee alone at 440 ppm protected the lemon flavor from the oxidation. The impact of the flavor and the balanced profile are partially protected (20%). The Lemon Candy note and the Citrus Fresh note are partially protected too (27% and 67% with rosemary, 27% and 33% with green bean coffee). However, the citrus note is not protected. Moreover, oxidized notes formation was limited. Rosemary at 30 ppm limited only Oxidised Citral Cherry note formation and Lime Distillate note formation (67% and 17%), but was not able to limit Oxidised Citral Animalic note formation and Oxidised Terpenes note formation. Green Bean Coffee at 440 ppm limited 17% of the Oxidised Citral Animalic note, Oxidised Terpenes note and Lime Distillate note formation, and 67% of the Oxidised Citral Cherry note formation.

Surprisingly, combining both extracts with a half lower dosage, we observed an important synergistic effect. The citrus note was not protected with extracts alone, whereas we observed 50% of protection with the combination. Moreover, Lemon Candy and Lemon Fresh notes were better protected with the combination (47% and 67% vs only 27% and 50% expected). Finally, oxidized notes formation was better limited than expected. We expected a limitation of 8% of the Oxidised Citral Animalic note and we obtained a limitation of 17%. We expected a limitation of 67% of the Oxidised Citral Cherry note and we obtained a limitation of 83%. We expected a limitation of 8% of the Oxidised Terpenes note and we obtained a limitation of 17%. Finally, we expected a limitation of 17% of the Lime Distillate note, and we obtained a limitation of 33%.

At the light of these results, we expect that the same synergistic effect could be observed between Green Bean Coffee and another Rosmarinic Acid-rich plant, as Lemon Balm.

TABLE 7
Protection of lemon flavour (Citral freshness) during aging: comparison of rosemary,
green coffee beans, and the composition containing rosemary and green coffee beans

| | | | | | Observed protection | | Expected | Observed |
|---|---|---|---|---|---|---|---|---|
| | Loss of flavour profile after | | | | of single | | protection of | protection of |
| | 4 weeks aging at 40° C. | | | | antioxidants (%) | | the blend (%) | the blend (%) |
| | | Rosemary | GBC | R + GBC | Rosemary | GBC | R + GBC | R + GBC |
| Lemon Flavour | | 30 | 440 | 15 + 200 | 30 | 440 | 15 + 200 | 15 + 200 |
| Profile | Control | ppm | ppm | ppm | ppm | ppm | ppm | ppm |
| Impact | 20% | 0% | 0% | 0% | 20% | 20% | 20% | 20% |
| Balanced | 60% | 40% | 40% | 40% | 20% | 20% | 20% | 20% |
| Citrus | 100% | 100% | 100% | 50% | 0% | 0% | 0% | 50% |
| Lemon Candy | 67% | 40% | 40% | 20% | 27% | 27% | 27% | 47% |
| Lemon Fresh | 100% | 33% | 67% | 33% | 67% | 33% | 50% | 67% |
| Oxidized Character | Increase of oxidized character | | | | | | | |
| Oxidized Citral | 17% | 17% | 0% | 0% | 0% | 17% | 8% | 17% |
| (animalic) | | | | | | | | |
| Oxidized Citral | 83% | 17% | 17% | 0% | 67% | 67% | 67% | 83% |
| (cherry) | | | | | | | | |
| Oxidized Terpenes | 50% | 50% | 33% | 33% | 0% | 17% | 8% | 17% |
| Lime Distillate | 67% | 50% | 50% | 33% | 17% | 17% | 17% | 33% |

1. An antioxidant composition comprising chlorogenic acid and one or more additional components or combination of components selected from a group consisting of:
(i) rosmarinic acid; and
(ii) phenolic diterpenes,
wherein the ratio of chlorogenic acid to the one or more additional components or combination of components is of between about 100:1 to about 1:100.

2. The composition according to claim 1, wherein the ratio of chlorogenic acid to the one or more additional components or combination of components is of between 50:1 to 1:50.

3. The composition according to claim 1, wherein the ratio of chlorogenic acid to rosmarinic acid is of between about 6:1 to about 1:6.

4. The composition according to claim 1, am wherein the ratio of chlorogenic acid to phenolic diterpenes is of between about 5:1 to about 1:5.

5. The composition according to claim 1, wherein the chlorogenic acid is provided as an extract of green coffee beans.

6. The composition according to claim 1, wherein the rosmarinic acid is provided as an extract of rosemary aerial parts and/or an extract of Melissa officinalis aerial parts.

7. (canceled)

8. A process for preventing and/or inhibiting oxidation of a food or beverage product for humans or animals, a nutritional supplement, a nutraceutical formulation, a fragrance or flavouring, a pharmaceutical or veterinary formulation, an oenological or cosmetic formulation, the process comprising the steps of contacting the food or beverage product for humans or animals, the nutritional supplement, the nutraceutical formulation, the fragrance or flavouring, the pharmaceutical or veterinary formulation, the oenological or the cosmetic formulation with the composition according to claim 1, wherein the food is a product selected from the group consisting of meat, beverages, sauces, patisseries, dairy products, oils, bakery, filling and edible ink.

9. (canceled)

10. (canceled)

11. (canceled)

12. A food or beverage product for humans or animals, a nutritional supplement, a nutraceutical formulation, a fragrance or flavouring, a pharmaceutical or veterinary formulation, an oenological or cosmetic formulation comprising the antioxidant composition according to claim 1 and a food or beverage product for humans or animals, a nutritional supplement, a nutraceutical formulation, a fragrance or flavouring, a pharmaceutical or veterinary formulation, an oenological or cosmetic formulation adjuvant.

13. The food or beverage product for humans or animals, nutritional supplement, a nutraceutical formulation, fragrance or flavouring, pharmaceutical or veterinary formulation, oenological or cosmetic formulation according to claim 12, wherein the composition in the foodstuff comprises a ratio of chlorogenic acid to phenolic diterpenes of between about 0.1:1 to about 10.1.

14. The food or beverage product for humans or animals, nutritional supplement, nutraceutical formulation, fragrance or flavouring, pharmaceutical or veterinary formulation, oenological or cosmetic formulation according to claim 12, wherein the composition in the foodstuff comprises between:
about 0.1% w/w to about 55.0% w/w of chlorogenic acid;
about 0.1% w/w of caffeine; and
at least about 0.1% w/w of phenolic diterpenes.

15. The product according to any claim 12, wherein the concentration of the composition in the product is of about 1 to about 10000 ppm, in respect to the final weight of the product.

16. The product according to claim 15, wherein the concentration of the composition in the product is of about 5 to 50 ppm, in respect to the final weight of the product.

17. The product according to claim 12, wherein the concentration of chlorogenic acid is from about 5 ppm to about 1000 ppm, in respect to the final weight of the product.

18. The product according to claim 12, wherein the concentration of phenolic diterpenes is from about 5 ppm to about 1000 ppm, in respect to the final weight of the product.

19. The product according to claim 12, wherein the concentration of rosmarinic acid is from about 5 ppm to about 1000 ppm, in respect to the final weight of the product.

20. The product according to claim 18, wherein the foodstuff is a meat product and the concentration of the composition in the foodstuff is of between 5 ppm to about 20 ppm of chlorogenic acid and of between about 5 ppm to about 25 ppm of phenolic diterpenes, in respect to the final weight of the product.

21. The product according to claim 20, wherein the foodstuff is a meat product and wherein the concentration of the composition in the foodstuff is of about 13.5 ppm of chlorogenic acid and of about 15 ppm of phenolic diterpenes, in respect to the final weight of the product.

22. The product according to claim 21, wherein the product is a meat product and wherein the concentration of the composition in the foodstuff is from about 5 ppm to about 15 ppm and from about 5 ppm to about 20 ppm of phenolic diterpenes, in respect to the final weight of the product.

23. The product according to claim 19, wherein the product is a beverage, wherein the concentration of the composition in the foodstuff is of between about 1 ppm to about 100 ppm of chlorogenic acid and of between about 0.1 ppm to about 50 ppm of rosmarinic acid, in respect to the final weight of the product.

24. The product according to claim 23, wherein the product is a beverage, wherein the concentration of the composition in the foodstuff is of about 9 ppm of chlorogenic acid and of about 1.5 ppm of rosmarinic acid, in respect to the final weight of the product.
